(19) United States
(12) Patent Application Publication
(10) Pub. No.: US 2006/0135618 A1
(43) Pub. Date: Jun. 22, 2006

(54) Medicine comprising a thiourea for use as depigmenting agent or anti-mutagenic and anti-carcinogenic agent

(76) Inventors: Daniel Jean, Vic-Le--comte (FR); Cherif Rabhi, Beaumont (FR); Veronique Schwaab, La Roche Blanche (FR)

Correspondence Address: STEPTOE & JOHNSON LLP, 1330 CONNECTICUT AVENUE, N.W., WASHINGTON, DC, 20036, US

(21) Appl. No.: 10/530,723

(22) PCT Filed: Oct. 3, 2003
(86) PCT No.: PCT/FR03/02907

§ 371 Date: Nov. 16, 2005

(30) Foreign Application Priority Data
Oct. 11, 2002 (FR) 02/12697

(51) Int. Cl. A61K 31/17

(52) U.S. Cl. 514/580

(57) ABSTRACT

The invention concerns a medicine or a cosmetic composition comprising at least one thiourea of general formula (I), or at least one of its monooxide or dioxide derivatives of general formulae (IIa), (IIb) and (III), or mixtures thereof. This medicine is advantageously used for inhibiting tyrosinase, inhibiting melanin synthesis, for lightening or depigmenting the skin or for eliminating age spots and as antimutagenic and anti-carcinogenic agent.

The present invention relates to medicinal products containing thioureas or their monoxide or dioxide derivatives, in particular having a depigmenting action, but also as an antimutagenic and/or anticarcinogenic agent.

Skin pigmentation in humans arises from a complex series of cellular processes that takes place in a unique population of cells called melanocytes. The melanocytes are located in the lower part of the epidermis, and their function is to synthesize a brown pigment, called melanin, which protects the body from the damaging effects of ultraviolet radiation. The melanin is deposited in the melanosomes, granules present inside the melanocytes. The melanosomes are expelled from the melanocytes and are transported to the surface of the skin by the keratinocytes, which assimilate the melanin contained in the melanosomes. The dark complexion of the skin is proportional to the amount of melanin synthesized by the melanocytes and transferred to the keratinocytes. In some cases, it is preferable to reduce or inhibit melanogenesis, for example to lighten the skin, to remove blemishes due to ageing or to reduce hyperactivity of the melanocytes.

Cosmetic compositions containing a peroxide such as hydrogen peroxide or zinc peroxide have long been used for the purpose of removing blemishes, such as patches of redness, that appear on the skin. However, the peroxides are extremely unstable, leading to problems in their storage. Moreover, stable incorporation of these peroxides in cosmetic bases is difficult and the peroxides themselves do not have a sufficient bleaching effect.

On the other hand, cosmetic preparations containing vitamin C, cysteine or colloidal sulphur have begun to be used for bleaching the skin. However, the effects of these substances are not satisfactory.

For a long time, hydroquinone has been the reference depigmenting molecule, and has been used in numerous cosmetic skin preparations. However, this product is not without danger and is very cytotoxic with respect to the melanocytes, possibly leading to irreversible depigmentation.

Recently, kojic acid has been used effectively as a substance for inhibiting the formation of melanin in human skin. Consequently, various cosmetic preparations intended for depigmenting the skin and containing kojic acid (publication of Japanese patent No. 56-18569) or an ester of kojic acid with an aromatic carboxylic acid such as cinnarnic acid or benzoic acid (publication of Japanese patent No; 60/100005) or of diesters of kojic acid (publications of Japanese patents No. 61-60801 and 60-17961) have been described. These kojic acids and esters of kojic acid are therefore known as being substances capable of inhibiting melanogenesis. However, the efficacy of kojic acid varies from one individual to another, and on average is insufficient.

The isothiocyariates and thiocyanates have also been described as depigmenting agents (WO 02/058664).

Accordingly, there is still an urgent need to find other depigmenting products.

To date, only the phenylthioureas have been described as possessing depigmenting activity (patent application US 2002/0044914). Some other thioureas, such as thiourea, phenylthiourea and dimethylthiourea, are known to be sensitizing (Contact and photocontact sensitivity problems associated with thiourea and its derivatives: A review of the literature and case reports. A. Dooms-Goosens et al., British Journal of Dermatology, 116, 4, 573-579). Furthermore, some of them, such as carbimazole, are used as antithyroid drugs.

Surprisingly, the applicants have discovered that certain molecules belonging to the thiourea family have a marked inhibitory effect on melanin synthesis in vitro.

Certainly, 1,3-bis-(5-methanesulphinylbutyl) thiourea is known to be one of the degradation products of sulphoraphane (an isothiocyanate possessing depigmenting activity: WO 02/058664) on prolonged contact with the skin, and especially with hot water by the following process: 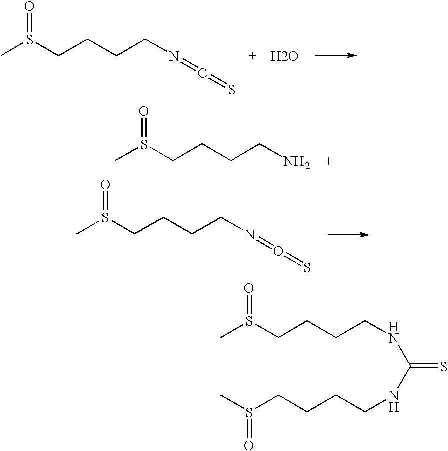
(Thermal degradation of sulforaphane in aqueous solution, Yi Jin, Mingfu Wang, Robert T. Rosen, and Chi-Tang-Ho. J. Agric. Food Chem. 1999, 47, 3121-3123). However, its pharmaceutical or cosmetic activity, especially as depigmenting agent, has never been described or suggested in the documents of the prior art, and it was not obvious that a degradation product of a depigmenting product would have greater activity than the said depigmenting product.

Mutagenesis occurs in the DNA and in the development of cells spontaneously or naturally, or as a secondary effect due to chemicals, high-energy radiation, stress, etc.

Mutagens are agents that cause such mutations. Often they are also carcinogenic (i.e. capable of inducing cancer). Present-day society is now aware and concerned by the presence of mutagens in the environment. Mutagens occur everywhere; some occur naturally in plants, many others are produced by burning organic materials (in particular during cooking), and others are produced by industry. Nowadays, the population is faced with many health problems. Many of these health problems result from lesions of the cells and DNA of the human body caused by mutagens, though also by mutagenesis factors, which contribute to increasing the lesions of the cells and DNA. These mutagens and mutagenesis factors include, among others: pollution, stress, ageing, cigarette smoking, ultraviolet light, excessive exercise, tissue lesions, etc. Among the diseases resulting from lesions of the cells and DNA, we may mention: ageing, blemishes from ageing, cancers, cataracts, dry skin, fatigue, skin cancers, stress-induced damage, and wrinkles.

Although pharmaceutical compositions are available for producing an antimutagenic activity inside the body, they sometimes have significant side-effects.

Consequently, there is a need to find a new product with an antimutagenic effect, for preventing mutagenesis inside the body, and an anticarcinogenic effect.

Surprisingly, the applicants discovered that certain molecules belonging to the thiourea family have a marked antimutagenic and anticarcinogenic effect both with respect to mutagenic substances and to UVB.
The present invention therefore relates to a medicinal product containing at least one thiourea of the following general formula I: 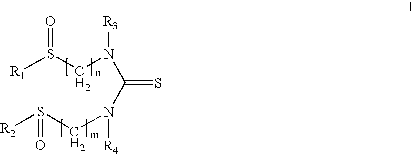
in which:
n is an integer between 1 and 12,
m is an integer between 1 and 12,
R₁, R₂, R₃ and R₄ represent, independently of one another, a hydrogen atom, a C₁-C₆ alkyl group or an aryl group,
or at least one of its monoxide or dioxide derivatives of the following general formulae IIa, IIb and III: 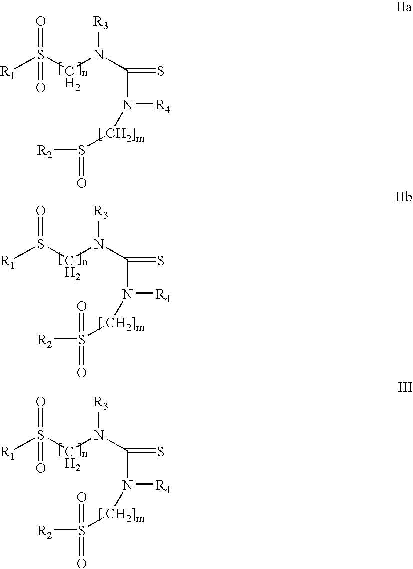
in which R₁, R₂, R₃, R₄, m and n are as defined above.
or their mixtures.

In the sense of the present invention, the term “C₁-C₆ alkyl group” means any alkyl group with from 1 to 6 carbon atoms, linear or branched, in particular the CH₃ group.

In the sense of the present invention, the term “aryl group” means one or more aromatic rings with 5 to 8 carbon atoms, which may be conjugated or fused. In particular, the aryl groups can be phenyl or naphthyl groups and can be substituted by halogen atoms, alkyl groups as defined above, the OH group or the nitro group.

Advantageously, the thioureas of general formulae I, IIa, IIb and III are such that the groups R₁ and R₂ are identical, R₃ and R₄ are identical and m=n. Even more advantageously, R₁=R₂═CH₃.
Advantageously m=n=4.
Advantageously R₃=R₄═H.
Even more advantageously, it comprises 1,3-bis-(5-methanesulphinylbutyl) thiourea, 1-(5-methanesulphinylbutyl)-3-(5-methanesulphonylbutyl) thiourea, or 1,3-bis-(5-methanesulphonylbutyl) thiourea of the following formulae:

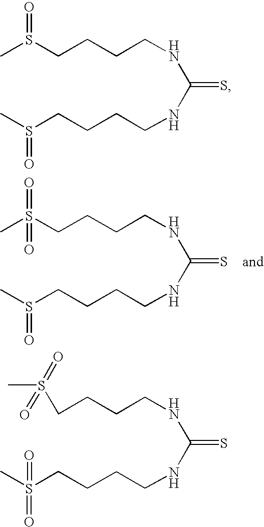

The thio ureas according to the present invention are either commercially available, or can be prepared, for example for 1,3-bis(5-methanesulphinylbutyl)-thiourea, by thermal degradation of sulphoraphane. In particular, the monoxide or dioxide derivatives can be obtained from the corresponding unoxidized thiourea by the action of an oxidizing agent, such as hydrogen peroxide for example.

Advantageously, the medicinal product according to the present invention can be used for inhibiting tyrosinase, in particular for reducing the action of tyrosinase by 50%, for inhibiting the synthesis of melanin, and for reducing hyperactivity of the melanocytes.

Advantageously, the medicinal product according to the present invention can be used as an antimutagenic agent, in particular against mutagenic substances and/or against UVB, and/or as an anticarcinogenic agent.

Advantageously, this medicinal product can prevent the appearance of cancers, especially cancers of the skin, blemishes from ageing, ageing, especially of the skin, and wrinkles.

The present invention also relates to cosmetic compositions containing at least one thiourea, according to the present invention, of the following general formula I: 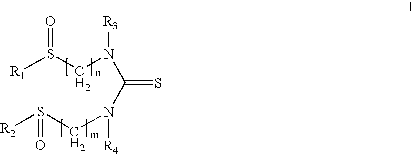
in which:
n is an integer between 1 and 12,
m is an integer between 1 and 12,
R₁, R₂, R₃ and R₄ represent, independently of one another, a hydrogen atom, a C₁-C₆ alkyl group or an aryl group,
or at least one of its monoxide or dioxide derivatives of the following general formulae IIa, IIb and III: 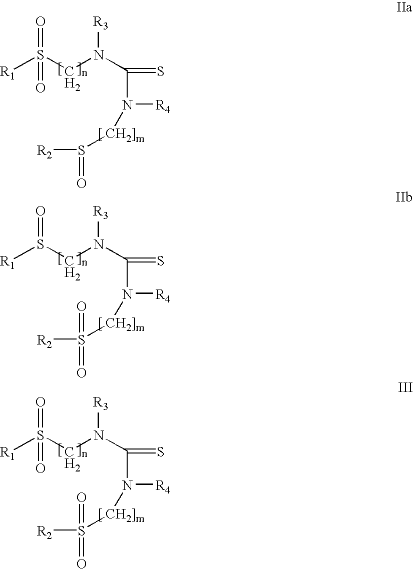
in which R₁, R₂, R₃, R₄, m and n are as defined above.
or their mixtures.

Advantageously, the said cosmetic composition is a depigmenting composition. It can be used for lightening, whitening or depigmenting the epidermis, removing blemishes from the skin, in particular from ageing or freckles, or preventing pigmentation of the epidermis. Advantageously, the said composition is intended for topical application.

Advantageously, the medicinal product or the cosmetic composition according to the present invention is in a form for oral or topical use, advantageously for topical use.
They can be in the forms that are commonly known for this type of administration, i.e. notably lotions, mousses, gels, dispersions, sprays, serums, masks, body lotions or creams for example, with excipients that in particular permit penetration of the skin to improve the properties and the accessibility of the active principle. These compositions generally contain, apart from the medicinal product or the cosmetic active according to the present invention, a physiologically acceptable medium, generally based on water or solvent, for example alcohols, ethers or glycols. They can also contain surfactants, preservatives, stabilizers, emulsifiers, thickeners, other active principles producing an additional or possibly synergistic effect, trace elements, essential oils, perfumes, colorants, collagen, chemical or mineral filters, moisturizers or hot-spring waters.

The present invention also relates to a cosmetic treatment of the skin by application of a cosmetic composition according to the present invention to the skin.

The following examples are given for illustration and are not limiting.

### EXAMPLE 1

### Synthesis of 1,3-bis-(5-methanesulphinylbutyl)-thiourea Starting from Sulphoraphane

### Synthesis of (D,L)-sulphoraphane:

Dissolve 40 g of 4-chlorobutyronitrile (ref. Aldrich C 3,000-0) in 800 ml of absolute ethanol previously distilled over sodium.
Then add 27 g of methane thioate (ref. Fluka 71742) and stir for 15 hours at 25° C. Filter the suspension on paper and evaporate under reduced pressure. Absorb in 400 ml of ethyl ether. Filter again on paper. An ethereal solution is obtained containing 32 g of raw 4-methylthiobutyronitrile.
Prepare a suspension of 25 g of lithium-aluminium hydride in 400 ml of ethyl ether.
Add the solution of 4-methylthiobutyronitrile gradually to the suspension of lithium-aluminium hydride, then reflux for 2 h 30 min.
Then neutralize the suspension by slowly adding, under reflux, 80 ml of distilled water. When boiling ceases, add 120 ml of distilled water to complete the neutralization of any hydride that remains. Filter on a glass frit. Wash the insoluble matter on the filter with 200 ml of ethyl ether. Combine the ethereal fractions and evaporate to dryness. 26.9 g of methylthiobutylamine is obtained. Absorb the product obtained in 80 ml of acetone, and then add, a little at a time, 23 ml of 35% hydrogen peroxide. Leave over night on a water bath at 50° C.
Then add a small amount of activated charcoal, filter and slowly add 200 ml of chloroform containing 20 ml of thiophosgene, then 300 ml of an aqueous solution of sodium hydroxide at 5%. Leave to act for 30 min.
Then extract the mixture in counter-current with 8 times 200 ml of dichloromethane. Collect the organic phase, dry over sodium sulphate and evaporate.
Then redistill the residue at 135° C. under 7.10⁻² torr. We obtain 12.5 g of D,L-sulphoraphane, the identity of which is verified by mass spectrometry.

### Synthesis of 1,3-bis-(5-methanesulphinylbutyl)-thiourea

Infrared spectra were obtained on a Perkin-Elmer 1600 F11R (neat).
Proton and carbon-13 NMR spectra were obtained on a Brucker AM 200 SY at 200 MHz for proton, 50.3 MHz for carbon. The chemical shifts are identical in parts per million (ppm) relative to the signal of deuterated chloroform CDCl₃ at 7.25 ppm for the proton and 76.9 ppm (central line of the deuterated chloroform) for carbon.
The mass spectra were obtained on a Normag/SIDAR V 2.3 by the methods of chemical ionization (NH₃) or electron impact.
   Chromatography
The reactions were monitored by thin-layer chromatography on plates of silica gel type 60F 254 (Merck, Art. 7735). The reagents and products were visualized in UV light then by treatment with a 10% ethanol solution of phosphomolybdic acid followed by heating.
Flash chromatography was carried out on silica gel ICN 60, 230-400 mesh.
   Distillation of the Solvents
The ethyl ether and THF were distilled under nitrogen on sodiui/benzophenone. The cyclohexane, ethyl acetate and methanol used for the chromatography were distilled before use.
   Method
Under an inert atmosphere, dilute 2 g (11 mmol) of sulphoraphane obtained above in 15 ml of water, then reflux the solution for 24 hours, protected from the light. At room temperature, concentrate the solution at reduced pressure and absorb the residue in dichloromethane for chromatography on a column of silica gel (eluent: CH₂Cl₂/CH₃OH: 90/10) to give 1.70 g (5 mmol) of 1,3-bis-(5-methanesulphinylbutyl)-thiourea (bis MSiBT) as a colourless oil, at a yield of 43%.
¹H NMR (300 MHz): 6.45 (d1, 2H, NH), 3.53 (t. H, J=6.0 Hz), 2.87 (m, 4H), 2.65 (s, 6H), 1.78 (m, 8H).
¹³C NMR (50.3 MHz): 24.0 (CH₂), 32.1 (CH₂), 41.2 (CH₂—CH₂), 47.2 (CH₃), 57.4 (CH₂SO), C (IV) not observed.
IR (ν, cm⁻¹): 2176, 2096, 1140, 940 cm⁻¹.
IC.MS m/z: 312 (MH⁺).

### EXAMPLE 2

### Preparation of 1-(5-methanesulphinylbutyl)-3-(5-methanesulphonylbutyl)-thiourea

Oxidation of just one of the two sulphur atoms of the sulphoraphane molecule was achieved by carrying out the reaction of example 1 of synthesis of 1,3-bis-(5-methanesulphinylbutyl)-thiourea in the open air, rather than under an inert atmosphere.
Dilute 1 g (5.5 mmol) of sulphoraphane in 10 ml of water, then reflux the solution for 24 hours, protected from the light.
At room temperature, concentrate the solution under reduced pressure and absorb the residue in dichloromethane for chromatography on a column of silica gel (eluent: CH₂Cl₂/CH₃OH: 90/10) to give 200 mg of 1-(5-methanesulphinylbutyl)-3-(5-methanesulphonylbutyl)-thiourea (MSBMSBT) as a colourless oil, at a yield of 12%.
¹H NMR (300 MHz): 6.55 (solid, 2H, NH), 3.55 (t, 4H, CH₂), 3.15 (t, 2H, SO₂CH₂), 2,80 (t, 2H, SOCH₂), 2.60 (s, 3H, SOCH₃), 2.90 (s, 3H, SO₂CH₃), 1.80-1.87 (m, 8H, CH₂).
¹³C NMR (50.3 MHz): 20.5 (CH₂), 28.5 (CH₂), 39.8 (CH₃SO), 41.0 (CH₃SO₂), 43.5 (CH₂—NH), 54.1 (CH₂SO), 44.8 (CH₂SO₂), 183.35 (C═S).
IR (ν, cm⁻¹): 2176, 2096, 1140, 940 cm⁻¹.
IC.MS m/z: 328 (MH⁺).

### EXAMPLE 3

### Preparation of 1,3-bis-(5-methanesulphonylbutyl) thiourea from 1-isothiocyanato-4-methylsulphonylbutane

1 g (5 mmol) of a solution of 1-isothiocyanato-4-methylsulphonylbutane in 15 ml of water is refluxed for 3 hours. At room temperature, concentrate the solution under reduced pressure and purify the residue by recrystallization from methanol to give 535 mg of 1,3-bis-(5-methanesulphonylbutyl) thiourea (bis MSoBT) as a beige solid, i.e. a yield of 40%.
¹H NMR (300 MHz): 6.50.(sl, H, NH), 3.55 (t, 4H, J =6.0 Hz), 2.75 (t, 4H, SO₂CH₂), 2.60 (s. 6H, SO₂CH₃), 1.70-1.90 (m, 8H, CH₂CH₂).
¹³C NMR (50.3 MHz): 21.0 (CH₂), 29.2 (CH₂), 38.5 (CH₃), 44.0 (CH₂—NH), 53.9 (CH₂SO), 182.6 (C═S).
IR (ν, cm⁻¹): 2168, 2096, 1144, 930 cm⁻¹.
IC.MS m/z: 344 (MH⁺).
Melting point: 142° C.

### EXAMPLE 4

### Preparation of bis (MSoBT) from bis MSiBT

Slowly add, at 0° C., 0.70 ml (2.2 equiv., 6.6 mmol) of 35% hydrogen peroxide to a solution of bis MSiBT (1 g, 3 mmol) in 10 ml acetone. Then leave the reaction medium to develop at room temperature for 40 hours. Eliminate the acetone by vacuum evaporation. Absorb the aqueous residue in chloroform, trapping the phase directly with sodium sulphate. Concentrate the organic phase under vacuum and purify the residue by recrystallization from methanol to give bis MSoBT as a beige solid at a yield of 30%.

### EXAMPLE 5

### Investigation of the Anti-Tyrosinase Effect of bis MSoBT in Comparison with Kojic Acid

Measurement of the Capacity for Inhibiting Tyrosinase:

Use the following reaction: oxidize L Dopa (L-3,4-dihydroxyphenylalanine, obtained from the company Sigma (ref. D-9626)), which is colourless, to the coloured dopachrome, which absorbs at 475 nm. This reaction is catalysed by fungal tyrosinase (EC 1.14.18.1, obtained from the company Sigma (ref. T-7755)). Record the kinetics of the reaction by measuring the optical density (O.D.) as a function of time at 30° C.
The various solutions used have the following compositions:
   Buffer pH 6.8:
Phosphate buffer 0.1M, pH 6.8
   Substrate Solution:
5 mM of L-DOPA in the buffer solution pH 6.8
   Solutions of Inhibitors:
The inhibitor molecules are dissolved directly in the buffer pH 6.8, in 50% methanol (methanol—distilled water) or in pure methanol, depending on their solubility.
The concentrations in weight per volume of the various solutions of inhibitors are: 0.2%, 0.1%, 0.05%, 0.025%, 0.0125%, 0.00625% and 0.00312%.
   Solution of Enzyme:
250 units of tyrosinase in the buffer solution pH 6.8.
The action of tyrosinase is evaluated from the initial reaction rate measured on the O.D. recordings.
Plot a curve of the initial reaction rates without inhibitors (concentration 0) and the rates at the different concentrations tested.
The inhibitory power of a molecule is defined as the concentration that reduces the action of tyrosinase by 50%.
Readings are taken for 3 min and the percentage inhibition is calculated (rate of inhibition and saturation plateau). bis MSoBT made as indicated above inhibits the enzymatic activity of tyrosinase by 50% at a final concentration of 8.3 mM. Kojic acid (obtained from the company Aldrich, ref. 22,046-9) shows the same percentage inhibition at a final concentration of 0.670 mM.
The low water-solubility of bis MSiBT and of MSBMSBT meant it was not possible to test these molecules for their anti-tyrosinase effect.

### EXAMPLE 6

### Investigation of the Depigmenting Effect of bis MSiBT, of MSBMSBT and of bis MSoBT in Comparison with Kojic Acid and Dimethylthiourea

The molecules bis MSiBT, MSBMSBT and bis MSoBT were also tested on melanin-producing human melanocyte-keratinocyte co-cultures, in order to test their depigmenting effect on a living system representing the melanization unit present in functioning human skin.
Kojic acid and dimethylthiourea were used as positive controls in this test.
Specimens of pigmented epidermis (black phenotype/co-culture of normal keratinocytes and melanocytes at the air/liquid interface) are obtained from and cultivated in accordance with the recommendations of MatteKcorporation, USA. The culture media are also supplied by this manufacturer. The products (35 mM kojic acid, 2.8 mM dimethylthiourea obtained from the company Aldrich, ref. D 18,870-0, 2.8 mM bis MSiBT obtained by the method described above, 2.8 mM MSBMSBT obtained by the method described above and 2.8 mM bis MSoBT obtained by the method described above) are applied in the culture medium for 21 days.
Each product is applied to four culture wells in solution in the culture medium. The depigmenting effect of the products is clearly visible since the control specimens of epidermis gradually become pigmented whereas the specimens of epidermis treated with the most active products display a pigmentation that is much less dark.
The depigmenting effect is evaluated by extraction of the melanin from the culture wells.

Extraction is carried out on a pool of two culture wells, which are homogenized in 0.45 ml of sodium dodecyl sulphate (SDS) at 1% containing 0.05 mM of EDTA and 10 mM of Tris HCl (amino-2-(hydroxymethyl)-2-propanediol-1,3), pH 6.8. Add 20 μl of proteinase K at 5 mg/ml to each homogenate. Digest over night at 45° C. Then add a further 20 μl of proteinase K and continue incubation for 4 hours. Then add 50 μl of 0.5 M solution of sodium carbonate and 10 μl of a 30% solution of hydrogen peroxide. Maintain the specimens at 80° C. for 30 min and then cool them. Extract the specimens with 100 μl of a chloroform-methanol mixture (2:1, v/v). After centrifugation at 10,000 g for 10 min, measure the optical density of the supernatant at 405 nm. The products are compared in Table 1 below (the results are expressed as percentage inhibition of the melanin in the co-cultures):

| Product tested | Concentration, mM | Inhibition % |
|---|---|---|
| Kojic acid | 35 | 32 |
| Dimethylthiourea | 2.8 | 36 |
| Bis MSiBT | 2.8 | 7.5 |
| MSBMSBT | 2.8 | 18 |
| Bis MSoBT | 2.8 | 29.6 |

Surprisingly, bis MSiBT displays a weaker depigmenting activity than MSBMSBT and especially bis MSoBT, the necessary concentration of which for obtaining approx. 30% inhibition is 25 times lower than for kojic acid, which is the reference molecule widely used in commerce for products intended for lightening the skin.
The products according to the present invention therefore possess a depigmenting activity.

### EXAMPLE 7

### Evaluation of the Antimutagenic Effect of bis MsoBT

This property was demonstrated in a modified version of the Ames test: the Vitotox 10kit (6400000) test of the company Thermolab Systems.
Generally speaking, the Vitotox test is based on bacteria containing the lux operon of Vibrio fisheri under the transcriptional control of the mutated recN promoter (controlled by the SOS system of the bacterium). After incubation of the bacteria with a genotoxic product, the recN promoter is derepressed and the lux operon is expressed: emission of light, the said emission being proportional to the genotoxicity of the product. Some products act directly on the production of light or increase the metabolism of the bacteria, creating false positives. Moreover, a bacterial strain possessing a constitutive lux operon is used as control. The same strain is used as cytotoxicity control (false negatives).

### EXAMPLE 7.1

### Evaluation of the Antimutagenic Effect of bis MsoBT Against MMS (methylmethanesulphonate)

In order to test the antimutagenic effect against genotoxic molecules, a genotoxic molecule, methylmethanesulphonate (MMS), is used, which is compared against the genotoxicity of a mixture of reference mutagenic molecules, 4-nitroquinoline oxide and benzo(a)pyrene. MMS is placed alone or mixed with bis MsoBT at increasing concentrations and the emissions of light are compared. It is then possible to define an inhibitory concentration 50 of mutagenesis.
Cultures of Salmonella typhimurium TA 104 recN2-4 (for testing genotoxicity) and of Salmonella typhimurium TA 104 prl (for testing cytotoxicity) in Nutrient Broth 8 g/L medium are effected then incubated at 37° C. with stirring over night (O.D. between 0.2 and 0.5 for the bacteria for testing genotoxicity and between 0.4 and 0.6 for the bacteria for testing cytotoxicity). On the next day, the bacteria for testing genotoxicity are diluted to 1/10 with the Nutrient Broth medium and to ½ for the bacteria for testing cytotoxicity.
A stock solution of MMS at 90 mM in H₂O is prepared and successive dilutions at ½ are made in a 96-well plate. The reference genotoxic products (4-nitroquinoline oxide (NQO)/benzo(a)pyrene (BAP)) are used at 0.4 ppm for NQO and 800 ppm for BAP. The test product is used with or without metabolic activation (fraction S9: microsomal homogenate containing cytochrome P450: 2.45 mL/mL final).
The kinetics of emission of light, as a function of the genotoxicity and of the cytotoxicity of the product, is measured for 3 hours (measurements every 5 minutes) with a Fluoroskan Ascent FL (bermnoLabsystems).
The products are supplied by the companies Sigma-Aldrich.
   Results:
bis MsoBT has an inhibitory concentration 50 of the mutagenic effect of MMS of 17 ppm without S9 and of 20 ppm with S9.

### EXAMPLE 7.2

### Evaluation of the Antimutagenic Effect of bis MsoBT Against UVB

To evaluate the antimutagenic effect against WVB, bacterial cultures are irradiated for a time that permits an average emission of 20 units of light (RLU). Then the bis MsoBT is placed at increasing concentrations in the culture medium before irradiation and an inhibitory concentration 50 is determined.
The procedure in example 1 is followed but without MMS, and irradiating the bacterial cultures for 20 s with WVB 254 nm.
In these conditions, MsoBT has an inhibitory concentration 50 of the mutagenic effect induced by UVB of 25 ppm.

1.-11. (canceled)

12. A pharmaceutical composition comprising a pharmaceutically effective amount of at least one thiourea of the following general formula I: 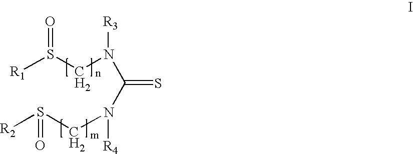
in which:
n is an integer between 1 and 12,
m is an integer between 1 and 12,
R₁, R₂, R₃ and R₄ represent, independently of one another, a hydrogen atom, a C₁-C₆ alkyl group, or an aryl group,
or at least one of its monoxide or dioxide derivatives of the following general formulae IIa, IIb and III: 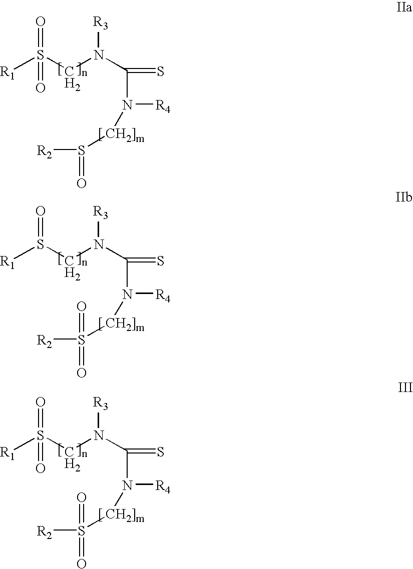
in which R₁, R₂, R₃, R₄, m and n are as defined above,
or their mixtures, and a pharmaceutically acceptable carrier therefor.

13. The pharmaceutical composition of claim 12, wherein the pharmaceutically effective amount is an amount effective to inhibit tyrosinase.

14. The pharmaceutical composition of claim 12, wherein the pharmaceutically effective amount is an amount effective to inhibit the synthesis of melanin.

15. The pharmaceutical composition of claim 12, wherein the pharmaceutically effective amount is an amount effective to reduce hyperactivity of melanocytes.

16. The pharmaceutical composition of claim 12, wherein the pharmaceutically effective amount is an antimutagenic and/or anticarcinogenic effective amount.

17. The pharmaceutical composition of claim 16 wherein the pharmaceutically effective amount is an amount effective for treating or preventing lesions of cells and DNA of the human body caused by mutagens and by mutagenis factors.

18. The pharmaceutical composition of claim 17, wherein the pharmaceutically effective amount is an amount effective to prevent the appearance of cancer.

19. The pharmaceutical composition of claim 18, wherein the pharmaceutically effective amount is an amount effective to prevent the appearance of skin cancer.

20. The pharmaceutical composition of claim 12, wherein R₁ and R₂ are identical, R₃ and R₄ are identical, and m=n.

21. The pharmaceutical composition of claim 12, wherein each of R₁ and R₂ is methyl.

22. The pharmaceutical composition of claim 12, wherein each of m and n is 4.

23. The pharmaceutical composition of claim 12, wherein each of R₃ and R₄ is H.

24. The pharmaceutical composition of claim 12, wherein the composition is in a form for topical use.

25. A depigmenting cosmetic composition comprising a depigmenting effective amount of at least one thiourea of the following general formula I: 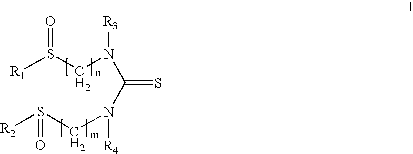
in which:
n is an integer between 1 and 12,
m is an integer between 1 and 12,
R₁, R₂, R₃ and R₄ represent, independently of one another, a hydrogen atom, a C₁-C₆ alkyl group or an aryl group,
or at least one of its monoxide or dioxide derivatives of the following general formulae IIa, IIb and III: 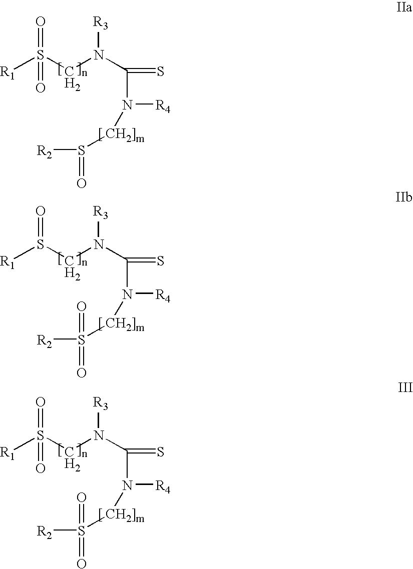
in which R₁, R₂, R₃, R₄, m and n are as defined above, or their mixtures, and a cosmetically acceptable carrier therefor.

26. A method of lightening, whitening or depigmenting the epidermis, removing skin blemishes, especially those caused by ageing or freckles, or preventing pigmentation of the epidermis comprising applying to a patient in need thereof an effective amount of the composition of claim 25.

27. A thiourea of the following general formula I: 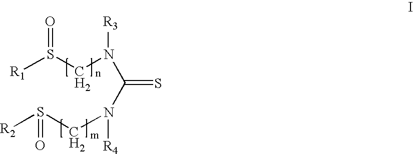
in which:
n is an integer between 1 and 12,
m is an integer between 1 and 12,
R₁, R₂, R₃ and R₄ represent, independently of one another, a hydrogen atom, a C₁-C₆ alkyl group, or an aryl group,
or at least one of its monoxide or dioxide derivatives of the following general formulae IIa, IIb and III: 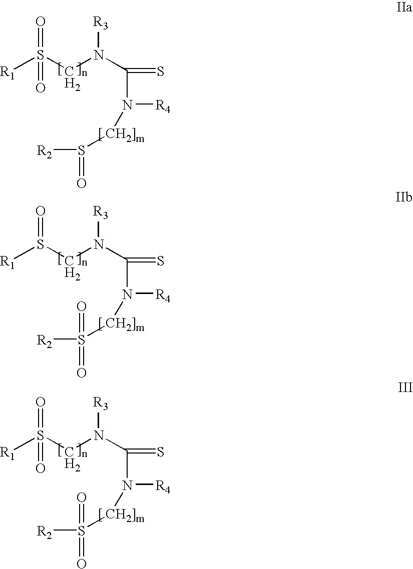
in which R₁, R₂, R₃, R₄, m and n are as defined above, with the exception of N, N-di(methylsulphinylbutyl)thiourea and dicheirolin thiourea.

28. A method of treating ageing, dry skin, fatigue and/or wrinkles comprising applying to a patient in need thereof an effective amount of the composition of claim 25.

29. A method of inhibition of tyrosinase comprising administering to a patient in need thereof a tyrosinase-inhibiting amount of the pharmaceutical composition of claim 12.

30. A method of inhibition of the synthesis of melanin comprising administering to a patient in need thereof a melanin synthesis-inhibiting amount of the pharmaceutical composition of claim 12.

31. A method of reduction of hyperactivity of melanocytes comprising administering to a patient in need thereof a melanocytes hyperactivity-reducing amount of the pharmaceutical composition of claim 12.

32. A method of treating or preventing lesions of cells and DNA of the human body caused by mutagens and by mutagenis factors comprising administering to a patient in need thereof a mutagen caused human body cell and DNA lesion treating or preventing amount of the pharmaceutical composition of claim 12.

33. A method of preventing the appearance of cancer comprising administering to a patient in need thereof a cancer-preventing amount of the pharmaceutical composition of claim 12.

34. A method of preventing the appearance of skin cancer comprising administering to a patient in need thereof a skin cancer-preventing amount of the pharmaceutical composition of claim 12.
